(19) United States
(12) Patent Application Publication
(10) Pub. No.: US 2018/0000542 A1
(43) Pub. Date: Jan. 4, 2018

(54) FLEXIBLE ELECTRODE

(71) Applicant: St. Jude Medical, Cardiology Division, Inc., St. Paul, MN (US)

(72) Inventors: Andrew Ryan Oliverius, Eagan, MN (US); James C. Marrs, Arden Hills, MN (US); Tim Marass, Minneapolis, MN (US)

(21) Appl. No.: 15/640,916

(22) Filed: Jul. 3, 2017

Related U.S. Application Data
(60) Provisional application No. 62/357,981, filed on Jul. 2, 2016.

(51) Int. Cl. A61B 18/14; A61B 18/00

(52) U.S. Cl. CPC A61B 18/1492; A61B 2217/007; A61B 2018/1475; A61B 2018/00964; A61B 2018/00821; A61B 2018/00184; A61B 2018/00791; A61B 2018/00773; A61B 2018/00214; A61B 2018/00815

(57) ABSTRACT

A flexible electrode includes an internal spring with an increased spring rate and/or distal-end and proximal-end turns having increased diameters. In an embodiment, a catheter electrode comprises an electrode body defining a cavity therein, the cavity comprising a first diameter; and a spring disposed within the cavity, the spring comprising a second diameter; wherein the first diameter is equal to the second diameter.

### CROSS-REFERENCE TO RELATED APPLICATION

This application claims priority to U.S. provisional patent application No. 62/357,981 entitled “Flexible electrode,” filed 2 Jul. 2016, which is hereby incorporated by reference as though fully set forth herein.

a. Field

The instant disclosure relates generally to a flexible tip electrode and to catheter tips incorporating such a flexible tip electrode.

b. Background Art

Electrophysiology catheters are used in a variety of diagnostic, therapeutic, and/or mapping and ablative procedures to diagnose and/or correct conditions such as atrial arrhythmias, including for example, ectopic atrial tachycardia, atrial fibrillation, and atrial flutter. Arrhythmias can create a variety of conditions including irregular heart rates, loss of synchronous atrioventricular contractions, and stasis of blood flow in a chamber of a heart, which can lead to a variety of symptomatic and asymptomatic ailments and even death.

Typically, a catheter is deployed and manipulated through a patient's vasculature to the intended site, for example, a site within a patient's heart or a chamber or vein thereof. The catheter carries one or more electrodes that can be used for cardiac mapping or diagnosis, ablation and/or other therapy delivery modes for example. Once at the intended site, treatment can include, for example, radio frequency (RF) ablation, cryoablation, laser ablation, chemical ablation, high-intensity focused ultrasound-based ablation, microwave ablation, and/or other ablation treatments. The catheter imparts ablative energy to cardiac tissue to create one or more lesions in the cardiac tissue and oftentimes a contiguous or linear and transmural lesion. This lesion disrupts undesirable cardiac activation pathways and thereby limits, corrals, or prevents errant conduction signals that can form the basis for arrhythmias.

Because RF ablation can generate significant heat, which if not controlled can result in excessive tissue damages, such as steam pop, tissue charring, and the like, it can be desirable to monitor the temperature of ablation electrode assemblies. It can also be desirable to include a mechanism to irrigate the ablation electrode assemblies and/or targeted areas in a patient's body with biocompatible fluids, such as saline solution. The use of irrigated ablation electrode assemblies can also prevent the formation of soft thrombus and/or blood coagulation, as well as enable deeper and/or greater volume lesions as compared to conventional, non-irrigated catheters at identical power settings.

The foregoing discussion is intended only to illustrate the present field and should not be taken as a disavowal of claim scope.

### BRIEF SUMMARY

In one embodiment, a catheter electrode comprises: an electrode body defining a cavity therein, the cavity comprising a first diameter; and a spring disposed within the cavity, the spring comprising a second diameter; wherein the first diameter is equal to the second diameter.

In another embodiment, a catheter electrode comprises: an electrode body, the electrode body defining a cavity therein; and a spring disposed within the cavity, wherein the spring has a spring rate of about 150 grams per inch or greater.

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1 is a schematic view of a catheter incorporating a deflectable catheter shaft section in accordance with an embodiment of the present disclosure.

FIG. 2 is a cross-sectional view taken along the central longitudinal axis of a flexible tip electrode in accordance with an embodiment of the present disclosure.

FIG. 3 is a cross-sectional view taken along the central longitudinal axis of a flexible tip electrode in accordance with an embodiment of the present disclosure.

FIG. 4 is a cross-sectional view taken along the central longitudinal axis of a flexible tip electrode in accordance with an embodiment of the present disclosure.

FIG. 5 is a cross-sectional view taken along the central longitudinal axis of a flexible tip electrode in accordance with an embodiment of the present disclosure.

FIG. 6 is a cross-sectional view taken along the central longitudinal axis of a flexible tip electrode in accordance with an embodiment of the present disclosure.

### DETAILED DESCRIPTION OF THE DISCLOSURE

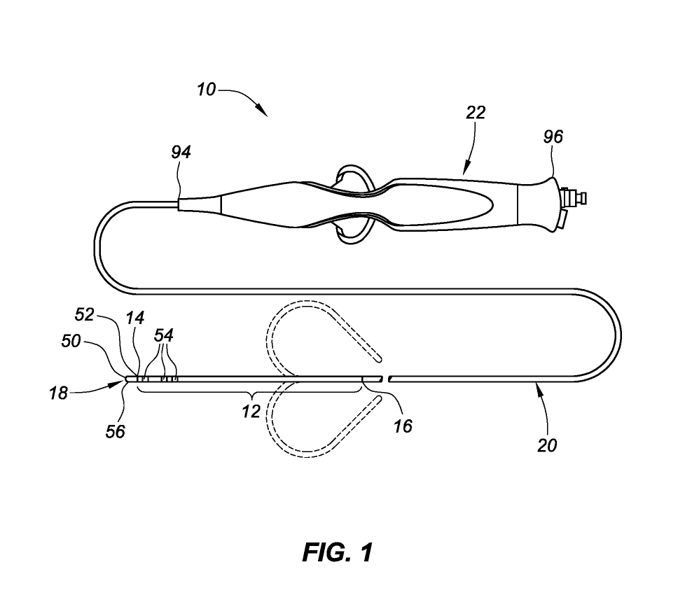

FIG. 1 generally illustrates an exemplary deflectable electrophysiology catheter 10 that comprises a deflectable catheter shaft section 12. The deflectable catheter shaft section 12 comprises an elongated body having a distal end 14 and a proximal end 16. In its most general form, the catheter 10 further comprises a tip assembly 18 located at the distal end 14 of the deflectable catheter shaft section 12, a proximal catheter shaft section 20 located at the proximal end 16 of the deflectable catheter shaft section 12, and a handle assembly 22. The catheter 10 may be used in any number of diagnostic and therapeutic applications, such as the recording of electrograms in the heart, the performance of a cardiac ablation procedure, and other similar applications/procedures.

Still referring to FIG. 1, the deflectable catheter shaft section 12 is disposed between the tip assembly 18 and the proximal catheter shaft section 20. The length and diameter of the deflectable catheter shaft section 12 can vary according to the application. Generally, the length of the deflectable catheter shaft section 12 can range from about 2 inches (50.8 mm) to about 6 inches (152.4 mm) and the diameter of the deflectable catheter shaft section 12 can range from about 5 French to about 12 French. The diameter of the deflectable catheter shaft section 12 can be about 7 French in accordance with some embodiments. Although these dimensions are mentioned in particular, the dimensions of the deflectable catheter shaft section 12 can vary in accordance with various applications of the deflectable catheter shaft section 12. The deflectable catheter shaft section 12 can be configured for deflection independent of the proximal catheter shaft section 20.

The tip assembly 18 (FIG. 1) comprises a tip electrode 56 having a distal end 50 and a proximal end 52. The tip electrode 56 may be configured for various functions and may include, without limitation, an active outer surface that is configured for exposure to blood and/or tissue. In an embodiment, the tip electrode 56 may be a flexible tip electrode comprising platinum iridium, gold, or stainless steel, for example. The tip electrode 56 may be affixed to the distal end 14 of the deflectable catheter shaft section 12 in a number of ways. For instance, the tip electrode 56 may be bonded to an inner radial surface of the deflectable catheter shaft section 12 using an epoxy material. As used herein, the term “radial surface” means a surface at a radial distance from a central axis or a surface developing uniformly around a central axis (for example, but without limitation, an arcuate surface, an annular surface, or a cylindrical surface). The tip electrode 56 of the tip assembly 18 may have a recess (not shown) formed therein that is sufficiently sized and configured to receive a wire (not shown) that is connected to the tip electrode 56. One end of the wire is connected to the tip electrode 56 and the other end is connected to, for example, monitoring or recording or ablation devices, such as a radiofrequency (RF) generator. The wire is typically a pre-coated wire that is insulated from other components in the tip assembly 18. The tip electrode 56 of the tip assembly 18 may further include a recess (not shown) formed therein that is configured to receive a thermocouple (not shown). The thermocouple may be configured to measure the temperature of the tip electrode 56, targeted tissue, and/or the interface therebetween and provide feedback to the monitoring or recording or ablation devices described hereinabove. The tip electrode 56 may further include a fluid lumen configured as a passageway for irrigation fluid.

The deflectable catheter shaft section 12 may also include one or more ring electrodes 54, as shown in FIG. 1. In an embodiment, one or more of the ring electrodes 54 may be a flexible electrode comprising platinum iridium, gold, or stainless steel, for example. The ring electrodes 54 may be used in place of or in addition to the tip electrode 56. For additional details, see U.S. patent application Ser. No. 13/704,619, filed on 16 Jun. 2011, titled “Catheter Having Flexible Tip With Multiple Flexible Segments,” incorporated by reference in its entirety as though fully set forth herein.

The proximal catheter shaft section 20 (FIG. 1) may also include one or more lumens (not shown). Generally, the proximal catheter shaft section 20 can include a single lumen. The proximal catheter shaft section 20 can also be constructed of a series of polymer layer(s) and braid structure(s). In particular, one or more wires woven together to form a cylindrical braid structure can substantially surround the one or more lumens of the proximal catheter shaft section 20. In addition, a polymeric material, such as polyurethane, nylon, or various types of plastic materials such as polyether block amides offered under the trademark PEBAX, or any other suitable material, can also substantially surround the one or more lumens of the proximal catheter shaft section 20. Regardless of the material used, the material must have capability to be displaced or to shrink when subjected to a process, such as for example, a heating process that is performed. The mechanical properties of the proximal catheter shaft section 20 can also be varied by varying the properties of the cylindrical braid structure(s) and the polymeric material (e.g., dimension of the cylindrical braid structure, number of wires comprising the braid, pattern of the braid, and/or durometers of the polymers). Additionally, the mechanical properties of the proximal catheter shaft section 20 can be varied along the length of the proximal catheter shaft section 20 in accordance with some embodiments of the disclosure or can be substantially constant along the entire length of the proximal catheter shaft section 20 in accordance with other embodiments of the disclosure.

The handle assembly 22 is coupled to the proximal catheter shaft section 20 at its proximal end (disposed within the handle assembly 22 and not shown). The handle assembly 22 is operative to, among other things, effect movement (i.e., deflection) of the deflectable catheter shaft section 12. The handle assembly 22 includes a distal end portion 94 and a proximal end portion 96.

The catheter 10 may include any number of other elements such as, for example and without limitation, thermocouples, thermistor temperature sensors, etc. for monitoring the temperature of targeted tissue and controlling the temperature. For additional details regarding the general construction of such a catheter, see U.S. patent application Ser. No. 14/213,289, filed on 14 Mar. 2014, titled “Flex Tip Fluid Lumen Assembly with Termination Tube,” incorporated by reference in its entirety as though fully set forth herein.

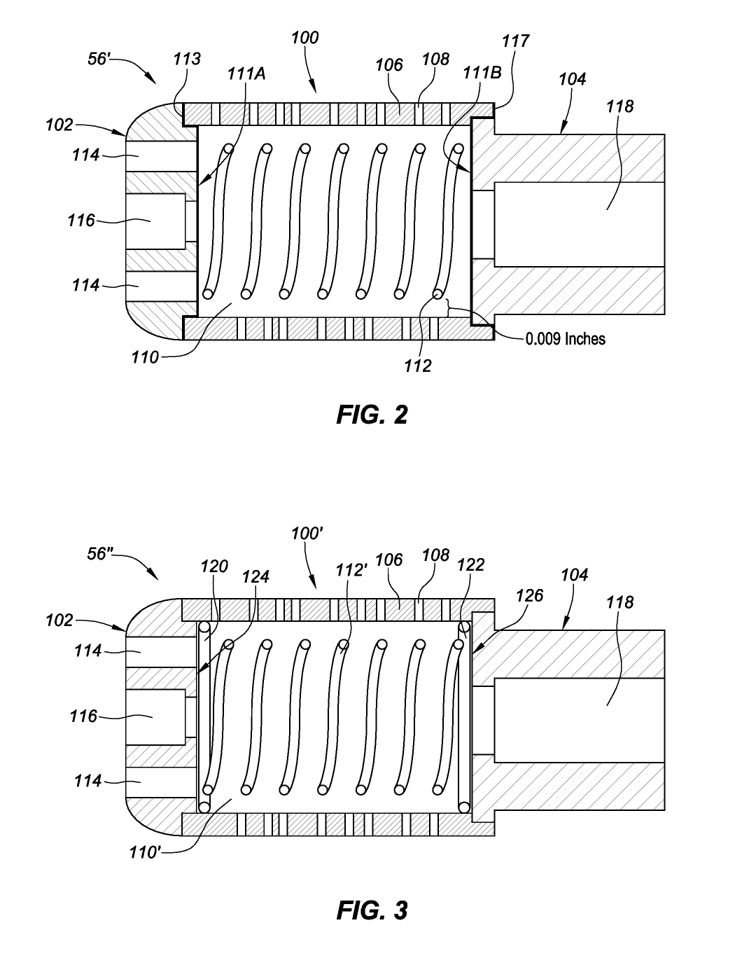

FIG. 2 illustrates an exemplary embodiment of a flexible tip electrode 56′. The flexible tip electrode 56′ includes three sections: an electrode body 100, an electrode cap 102, and a proximal stem 104. The electrode body includes an electrode wall 106 interspersed with a plurality of gaps 108 (e.g., linear gaps). The linear gaps 108 can extend through the electrode wall 106, as shown, allowing irrigant delivered to a center cavity 110 of the flexible tip electrode 56′ to pass through the electrode wall 106. The center cavity 110 includes a distal end 111A and a proximal end 111B. The electrode wall 106 may jut out slightly past the distal end 111A and proximal end 111B of the center cavity 110, as shown. The electrode body 100 further includes an internal spring 112, comprising a plurality of helical turns. The spring 112 is configured to be located within the center cavity 110 of the flexible tip electrode 56′ and to bias the flexible tip electrode 56′ into pre-determined arrangements. For example, the spring 112 can bias the flexible tip electrode 56′ in a longitudinal direction (e.g., by pushing the electrode cap 102 away from the proximal end 111B of the center cavity 110) or in a pre-bent configuration. In one embodiment, the spring 112 can comprise a resilient material such as stainless steel and/or nitinol, the latter also being a shape memory material. The spring 112 typically has an outer diameter that is smaller than the diameter of the center cavity 110 (e.g., approximately 0.009 inches smaller).

The electrode cap 102 of the flexible tip electrode 56′ may be coupled to an annular ledge or seat 113 connected to the distal end 111A of the center cavity 110 and to the electrode wall 106 by way of adhesive, epoxy, reflowed shaft polymer material, and/or other bonding materials or techniques. The electrode cap 102 includes one or more irrigation ports 114 (two are shown in FIG. 2), which may be evenly distributed around a longitudinal axis of the flexible tip electrode 56′. The electrode cap can further comprise a cap through-hole 116, which may be used for placement of a sensor or other desired material within the electrode cap 102. In an embodiment, a thermocouple or thermistor can be placed within the through-hole 116 and secured with a thermally conductive material. In other embodiments, the sensor can be coupled to the electrode cap 102 with a thermally-conductive and electrically-conductive material. In another embodiment, the through-hole 116 can be fluidly coupled to the electrode cavity and fluid can flow therethrough. For additional details, see U.S. patent application Ser. No. 14/724,169, filed on 28 May 2015, titled “Flex Tip Fluid Lumen Assembly with Thermal Sensor,” incorporated by reference in its entirety as though fully set forth herein.

The proximal stem 104 (FIG. 2) of the flexible tip electrode 56′ may be coupled may be coupled to an annular ledge or seat 117 connected to the proximal end 111B of the center cavity 110 and to the electrode wall 106 by way of adhesive, epoxy, reflowed shaft polymer material, and/or other bonding materials or techniques. Additionally, the proximal stem 104 can couple the flexible tip electrode 56′ to the deflectable catheter shaft section 12 (see FIG. 1) by way of adhesive, epoxy, reflowed shaft polymer material, and/or other bonding materials or techniques. Further, the proximal stem 104 can include a fluid lumen 118 for the flow of irrigant. The fluid lumen 118 may extend into the center cavity 110 of the electrode body 100.

The flexible tip electrode 56″ shown in FIG. 3 is similar to the flexible tip electrode 56′ of FIG. 2, with the exception that the spring 112′ of the flexible tip electrode 56″ includes two additional helical turns forming the distal and proximal ends of the plurality of helical turns: a distal-end turn 120 and a proximal-end turn 122. In an embodiment, both the distal-end turn 120 and the proximal-end turn 122 have outer diameters that match the diameter of the center cavity 110′ of the electrode body 100′. It should be noted that, in some embodiments, the entire spring may have an outer diameter that matches the diameter of the center cavity 110′. The distal-end turn 120 may be shaped to present an abutting contact surface 124 with the electrode cap 102. Likewise, the proximal-end turn 122 may be shaped to present an abutting contact surface 126 with the proximal stem 104. The contact surfaces 124 and 126 can be welded to the electrode cap 102 and the proximal stem 104, respectively, such as by a laser weld, for example. This results in a strengthened flexible electrode tip 56″ (compared to the flexible electrode tip 56′) without compromising function.

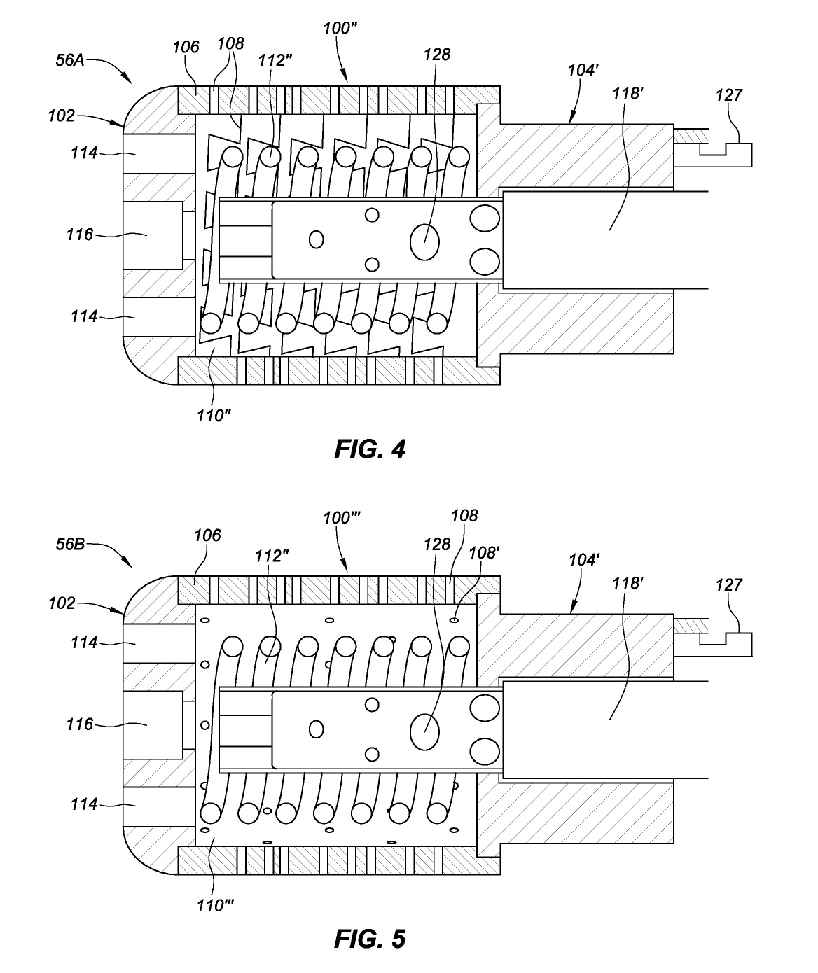

In order to facilitate integration of the flexible tip electrode 56′ of FIG. 2 with force-sensing technology, such as the TactiCath® contact force-sensing ablation catheter owned by St. Jude Medical, Inc., the present inventors have designed a modified the flexible tip electrode 56A, 56B (shown in FIGS. 4 and 5) with increased stiffness and decreased flexibility to provide accurate force measurements and retain the benefits of the flexible tip electrode 56′ (i.e., superior irrigation and lesion performance compared to standard non-irrigated or solid-irrigated tip electrodes). A spring of a more flexible tip electrode, such as the spring 112 of the flexible tip electrode 56′ shown in FIG. 2, may comprise a stainless steel wire with an outer diameter of about 0.004 inches and a spring rate of about 75 grams per inch (i.e., it takes about 75 grams of force to compress/deflect the spring one inch). Increasing the spring rate to about 150-400 grams per inch by using a steel wire with an outer diameter of about 0.005-0.008 inches, for example, results in a stiffer spring, such as a spring 112″ shown in FIGS. 4 and 5.

Referring to FIG. 4, the modified flexible tip electrode 56A includes an electrode body 100″, an electrode cap 102, and a proximal stem 104′, similar to the flexible electrode tip 56′ depicted in FIG. 2. The spring 112″ is formed from a wire with a diameter that is approximately twice that of the wire diameter for the spring 112 shown in FIG. 2, resulting in an increased spring rate and increased stiffness of the spring 112″. In effect, the increased spring rate of the spring 112″ prevents the flexible tip electrode 56A from flexing as much as a more flexible electrode, such as that described above including a spring with a 75 grams per inch spring rate, as the force required to flex it would be higher than typical outside forces seen in a clinical setting. Thus, the flexible tip electrode 56A is no longer “flexible” in the clinical setting. The loss of flexibility reduces the error in force calculations, but the flexible tip design still retains the benefits of directional irrigation and superior lesion performance.

The center cavity 110″ of the modified flexible tip electrode 56A includes a plurality of linear gaps 108, shown here as extending around the circumference of the electrode wall 106 of the flexible tip electrode 56A. The plurality of linear gaps 108 can form a variety of patterns on the electrode wall 106, such as the interlocking dovetail pattern of liner gaps 108 shown in FIG. 4 or the evenly distributed pattern of holes 108′ shown in FIG. 5. Alternatively, other gap/hole patterns may be used. As discussed above, the gaps or holes allow irrigant delivered from the fluid lumen 118′ to the center cavity 110″ of the flexible tip electrode 56A to pass through the electrode wall 106. This results in directional irrigation (i.e., the irrigant flows toward the distal end of tip electrode), which helps to improve tip cooling, as well as to prevent steam pops and other adverse events resulting from ablation. In addition, directional irrigation can allow for a therapeutically sufficient amount of irrigant to be delivered to the tissue, while avoiding irrigant overload.

As shown FIGS. 4 and 5, the fluid lumen 118′ can extend into the electrode body 100″. Additionally, the fluid lumen 118′ can comprise a plurality of side holes 128 in a distal section. The plurality of side holes 128 can be configured to deliver irrigant into the center cavity 110″ in a desired manner. In some embodiments, more proximally located side holes 128 can be larger in diameter than the side holes 128 found more distally on the fluid lumen 118′. This can help to keep fluid pressure relatively constant as irrigant flows out of the modified flexible tip electrode 56A. In other embodiments, the plurality of side holes 128 can comprise the same general diameter.

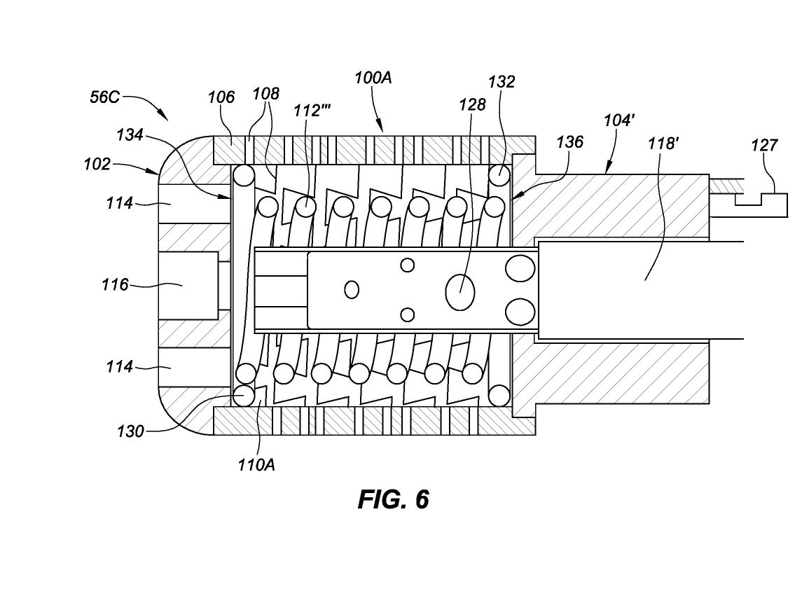

It should be noted that in the embodiments described with respect to FIGS. 4 and 5 above, as well as FIG. 6 below, a force sensor 127 can be located proximal to the proximal stem 104′, as shown. In other embodiments, the force sensor 127 may replace the proximal stem 104′. For additional details regarding the force sensor, see U.S. patent application Ser. No. 11/237,053, filed on 28 Sep. 2005, titled “Medical Apparatus System Having Optical Fiber Load Sensing Capability,” incorporated by reference in its entirety as though fully set forth herein.

The modifications to the flexible tip electrode shown and described in FIG. 3 and FIG. 4 or 5 can be combined, as shown in FIG. 6. The modified flexible tip electrode 56C is similar to that of FIG. 4 with the addition of a distal-end turn 130 and a proximal-end turn 132 to the internal spring 112′″. As in the embodiment shown in FIG. 3, both the distal-end turn 130 and the proximal-end turn 132 have outer diameters that match the diameter of the center cavity 110A of the electrode body 100A. Matching the outer diameter of the distal-end and proximal-end turns 130, 132 to the diameter of the center cavity 110A of the electrode body 100A provides a contact surface 134 between the spring 112′″ and the electrode cap 102, and a contact surface 136 between the spring 112′″ and the proximal stem 104, respectively. The contact surfaces 134 and 136 can be welded together, such as by a laser weld, for example. This allows the flexible electrode tip 56C is be both strengthened and stiffened, allowing for accurate contact force measurements, directional irrigation, and improved lesion performance.

Although several embodiments have been described above with a certain degree of particularity, those skilled in the art could make numerous alterations to the disclosed embodiments without departing from the present disclosure. It is intended that all matter contained in the above description or shown in the accompanying drawings shall be interpreted as illustrative only and not limiting. Changes in detail or structure may be made without departing from the present teachings. The foregoing description and following claims are intended to cover all such modifications and variations.

Various embodiments are described herein of various apparatuses, systems, and methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” “an embodiment,” or the like, means that a particular feature, structure, or characteristic described in connection with the embodiment is included in at least one embodiment. Thus, appearances of the phrases “in various embodiments,” “in some embodiments,” “in one embodiment,” “in an embodiment,” or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A catheter electrode comprising:
an electrode body defining a cavity therein, the cavity comprising a first diameter; and
a spring disposed within the cavity, the spring comprising a second diameter;
wherein the first diameter is equal to the second diameter.

2. The catheter electrode of claim 1, wherein the electrode body is flexible.

3. The catheter electrode of claim 1, wherein the first diameter comprises a diameter of the cavity.

4. The catheter electrode of claim 1, wherein the spring comprises a plurality of turns, wherein the plurality of turns comprises a distal-end turn and a proximal-end turn, and wherein the second diameter comprises an outer diameter of at least one of the distal-end turn and the proximal-end turn.

5. The catheter electrode of claim 4, further comprising:
a distal cap portion adjacent to the distal-end turn; and
a proximal stem portion adjacent to the proximal-end turn.

6. The catheter electrode of claim 5, wherein the distal-end turn and the distal cap portion are welded together to form a first contact surface; and wherein the proximal-end turn and the proximal stem portion are welded together to form a second contact surface.

7. The catheter electrode of claim 1, wherein the catheter tip electrode is irrigated.

8. The catheter electrode of claim 1, further comprising a thermocouple or thermistor.

9. The catheter electrode of claim 1, wherein the spring has a spring rate of about 150 grams per inch or greater.

10. A catheter electrode comprising:
an electrode body, the electrode body defining a cavity therein; and
a spring disposed within the cavity, wherein the spring has a spring rate of about 150 grams per inch or greater.

11. The catheter electrode of claim 10, wherein the spring has a spring rate of about 400 grams per inch or less.

12. The catheter electrode of claim 10, wherein the spring comprises a steel wire with a diameter of at least about 0.005 inches.

13. The catheter electrode of claim 10, further comprising:
an electrode wall surrounding the cavity; and
a plurality of gaps extending around an outer circumference of the electrode wall, the plurality of gaps configured to allow irrigant within the cavity to pass through the electrode wall.

14. The catheter electrode of claim 13, wherein the plurality of gaps are linear gaps.

15. The catheter electrode of claim 14, wherein the linear gaps form an interlocking dovetail pattern.

16. The catheter electrode of claim 13, wherein the plurality of gaps are circular holes.

17. The catheter electrode of claim 16, wherein the circular holes are evenly distributed around the outer circumference of the electrode wall.

18. The catheter electrode of claim 13, wherein the plurality of gaps are configured to permit directional flow of irrigation fluid.

19. The catheter electrode of claim 10, further comprising:
a distal cap portion adjacent to a distal-end surface of the electrode body; and
a proximal stem portion adjacent to a proximal-end surface of the electrode body.

20. The catheter electrode of claim 19, wherein the distal-end surface of the electrode body and the distal cap portion are welded together to form a first contact surface; and wherein the proximal-end surface of the electrode body and the proximal stem portion are welded together to form a second contact surface.

21. The catheter electrode of claim 10, further including a contact force sensor.

22. The catheter tip electrode of claim 10, further comprising a thermocouple or thermistor.
